(19) United States
(12) Patent Application Publication
(10) Pub. No.: US 2017/0367416 A1
(43) Pub. Date: Dec. 28, 2017

(54) DEODORIZING FILTER FOR MASK AND DEODORIZING MASK

(71) Applicant: TOAGOSEI CO., LTD., Tokyo (JP)

(72) Inventor: Yoshinao YAMADA, Nagoya-shi, Aichi (JP)

(73) Assignee: TOAGOSEI CO., LTD., Tokyo (JP)

(21) Appl. No.: 15/534,221

(22) PCT Filed: Oct. 28, 2015
(86) PCT No.: PCT/JP2015/080368

§ 371 Date: Jun. 8, 2017

(30) Foreign Application Priority Data
Dec. 17, 2014 (JP) 2014-254764

(51) Int. Cl. A41D 13/11; B01J 20/18; A62B 18/02; A61L 9/16; B01J 20/06; A61L 9/01; B01J 20/22

(52) U.S. Cl. CPC A41D 13/11; B01J 20/06; B01J 20/18; A61L 9/01; A62B 18/02; A61L 9/16; B01J 20/22

(57) ABSTRACT

An object of the invention is to provide a deodorizing filter superior in air-permeability as well as in deodorizing performance with respect to an unpleasant malodorous gas. A further object is to provide such a deodorizing filter and a deodorizing mask that do not have an unpleasant odor in themselves, or even after storage in a hermetically closed environment do not develop unpleasant odor or discoloration, and may be used comfortably. A deodorizing filter for a mask of the invention is provided with 2 or more layers of deodorizing fiber layers including a fiber and a chemisorption-type deodorizer, in which the deodorizing fiber layer contains a polyethylene resin fiber, and the thickness of the deodorizing fiber layer is from 0.15 to 0.4 mm, and the basis weight of the deodorizing fiber layer is from 20 to 45 g/m².

### TECHNICAL FIELD

The invention relates to a deodorizing filter for a mask and a deodorizing mask using the same.

### BACKGROUND ART

A mask for preventing a malodorous gas, a dust, a bacterium, a virus, etc. from entering into a respiratory organ has been heretofore used. Especially a mask against a malodorous gas contains in general a deodorizer for adsorbing a malodorous component, and for example a mask provided with a deodorizing fiber layer constituted with fibers, on which surface a deodorizer is adhered, formed into a sheet, or with a deodorizing fiber layer constituted with fibers, from which surface part of a deodorizer is exposed, formed into a sheet has been known. For example, Japanese Patent Application Laid-Open (JP-A) No. 2011-125596 describes a filter for a mask using an activated carbon sheet as a filter. Further, Japanese Unexamined Utility Model Application Publication (JP-Y) No. H05-33743 describes a deodorant mask mounting an air-permeable material containing both 1, or 2 or more kinds of metals selected from Fe, Mn, Al, Zn, and Cu, and a reaction product of the metal with an oxy-polybasic acid at an air-permeable part of the mask.

A filter for a mask with a plurality of filters layered one on another has been proposed for the sake of trapping a malodorous component. For example, JP-A No. H05-115572 describes a mask laminating 1 or more sheets of a filter material for a mask, which is constituted with particles of a specific calcium phosphate compound supported on a sheet-formed organic macromolecular substance, and has a large number of minute air-communicating pores. JP-A No. 2009-201634 discloses a mask formed by laminating the first nonwoven fabric constituted with fibers supporting calcium phosphates and the second nonwoven fabric using a nonwoven fabric supporting copper.

Meanwhile, a chemisorption-type deodorizer, which can exert excellent deodorizing performance even in a small amount, has been developed (JP-A No. 2000-279500, JP-A No. 2002-200149, and JP-A No. 2011-104274).

### DISCLOSURE OF THE PRESENT INVENTION

### Problems that the Present Invention is to Solve

Since a chemisorption-type deodorizer captures an odor by a reaction, it has an effect of deodorization in a short time. However, an objective malodor for a mask is in a gas state, and therefore a contact duration between a deodorizer and a malodorous gas is momentary. Since a nonwoven fabric supporting a deodorizer is also air-permeable, part of a malodorous gas always passes the nonwoven fabric without contacting a deodorizer therein. Therefore, a mask eliminating an odor to a degree that a malodor is almost not any more perceivable has not been materialized. Since a demand for comfortability has been intensified recently, a mask with such high deodorizing performance, that an uncomfortable feeling be suppressed through efficient adsorption of a malodorous gas, has been sought-after.

Although an activated carbon sheet as described in JP-A No. 2011-125596 has a high air-permeability, activated carbon is a physical adsorption-type deodorizer and adsorption and desorption of a malodorous component is reversible and the desorption speed is high. Therefore a sufficient deodorizing effect cannot be obtained, and the sheet is not adequate for use as a filter for adsorbing a malodorous component. Further, it has a drawback that a gas containing a malodorous component is re-released during a continuous use. With respect to a deodorant material described in JP-Y No. H05-33743, since a sufficient deodorizing effect is not obtained and a binder is not used, it is difficult to place the same in a large amount at an air-permeable part and the effect may be decreased due to uneven distribution of a deodorizer.

JP-A No. H05-115572 or JP-A No. 2009-201634 describes lamination of a plurality of deodorizing filters, however, the deodorizing performance with respect to a malodorous gas was still insufficient, and decrease in air-permeability due to lamination of deodorizing filters was significant.

In this regard, a fiber product, such as a deodorizing filter and a deodorizing mask, is in general packed (stored) in a packaging, such as a bag and a box made of paper, resin film, etc., after production, and in the case of fiber products obtained by sticking a deodorizer to a fiber surface using a binder, some may emit an unpleasant odor (unusual smell) derived from an unknown source among a deodorizer, a fiber and a binder constituting the fiber product, or a packaging material, when the same is taken out from a packaged state.

The invention was made in view of the recent situation described above with an object to provide a deodorizing filter superior in air-permeability as well as in deodorizing performance with respect to an unpleasant malodorous gas. Further, the invention has an object to provide such a deodorizing filter and a deodorizing mask that do not have an unpleasant odor in themselves, or even after storage in a hermetically closed environment do not develop unpleasant odor or discoloration and may be used comfortably. Solution to Problem

The invention is related to a deodorizing filter for a mask comprising 2 or more layers of deodorizing fiber layers comprising a fiber and a chemisorption-type deodorizer, wherein the deodorizing fiber layer contains a polyethylene resin fiber, and the thickness of each deodorizing fiber layer is from 0.15 to 0.4 mm, and the basis weight of the deodorizing fiber layer is from 20 to 45 g/m². It is additionally related to a deodorizing mask comprising as a laminate the deodorizing filter for a mask and a filter other than the deodorizing filter.

The term “malodorous component” means herein a substance causing a malodor, and a gas containing such a malodorous component is termed “malodorous gas”. The unit “ppm” with respect to a gas concentration is “ppm by volume”. “Air permeability” is an air permeability measured by a Frazier type method according to JIS L1096.

### Advantageous Effects of Invention

A deodorizing filter for a mask of the invention has sufficient air-permeability from one side to the other side, and exhibits superior deodorizing performance with respect to an unpleasant malodorous gas. Therefore, it can reduce a malodorous component in an atmosphere through use as a filter for adsorbing a malodorous component contained in a malodorous gas, such as an excretion odor, a putrid odor, and a tobacco odor.

Further, a deodorizing filter for a mask of the invention and a deodorizing mask using the same have no unpleasant odor in themselves, and therefore any unpleasant odor is not substantially emitted therefrom even when stored in a tightly closed environment, and discoloration is also suppressed, so that comfortable use becomes possible. A deodorizing mask of the invention is suitable for use at a place where a malodorous gas is generated (a medical job site, a nursing job site, an excretion spot, a sewage treatment plant, a waste treatment plant (incineration plant), a fertilizer plant, a chemical plant, a livestock farm, a fishing port, animal-related facilities, etc.)

### BRIEF DESCRIPTION OF THE DRAWINGS

FIG. 1: FIG. 1 is a schematic frontal view of an example of a deodorizing mask of the invention.

FIG. 2: FIG. 2 is a schematic sectional view of an example of a deodorizing mask of the invention.

### BEST MODE FOR CARRYING OUT THE PRESENT INVENTION

1. Deodorizing filter for mask

A deodorizing filter for a mask of the invention is a deodorizing filter comprising 2 or more layers of deodorizing fiber layers comprising a fiber and a chemisorption-type deodorizer, and having air-permeability across a deodorizing fiber layer from one side to the other side. A deodorizing filter of the invention may be used adapting to a targeted dimension or shape (planar structure, and spatial structure such as a pleat).

A deodorizing fiber layer constituting a deodorizing filter for a mask of the invention is preferably a fiber assembly containing at least one kind selected out of a conjugated fiber, in which a chemisorption-type deodorizer is buried in a surface of a substrate of a fiber such that the deodorizer is exposed, and a conjugated fiber, to which surface a chemisorption-type deodorizer is bonded by an adhesive layer. In this regard, the average diameter of a fiber such as a conjugated fiber contained in a fiber assembly is preferably from 5 to 30 μm, and more preferably from 10 to 25 μm.

A base material constituting a deodorizing fiber layer and a deodorizing filter may be made of either of a woven fabric and a nonwoven fabric, but is preferably made of a nonwoven fabric, because regulation to a desired thickness is easy, the production cost is low, and a control of the air-permeability is easy.

As a resin composing a fiber contained in a nonwoven fabric, a polyethylene resin is required to be included, because the adhesion with a chemisorption-type deodorizer and air-permeability are obtained sufficiently and a deodorizing filter itself does not emit an unpleasant odor. Examples of other resin include polypropylene, polyester, poly(vinyl chloride), poly(acrylic acid), polyamide, poly(vinyl alcohol), polyurethane, polyvinyl ester, polymethacrylate, and rayon. When a polyethylene resin is used as a mixture with another resin, the content of a polyethylene resin is preferably from 10 to 100 mass % with respect to the total resin, more preferably from 20 to 90 mass %, and yet more preferably from 30 to 80 mass %. As for a nonwoven fabric, a nonwoven fabric in which entanglements are created by a needle punching method or a hydroentangling method, a nonwoven fabric produced by a thermal bonding method, and a nonwoven fabric produced by a spunbond method are preferable.

As a deodorizer for a malodorous gas, a type which adsorbs a malodorous component by physical adsorption such as activated carbon, and a type which degrades a malodorous component during contact such as a photocatalyst, besides a type which adsorbs a malodorous component by chemisorption, or forms a chemical bond with a malodorous component, such as a chemisorption deodorizer of the invention, are common. However, when a deodorizer is used as a filter for letting a malodorous gas pass, it is necessary to adsorb a malodorous component in a short time during which a malodorous gas passes, and therefore a physical adsorption type, with which a malodorous gas is re-released during a continuous use, or a degradation type with which degradation occurs by irradiation with light, is not able to exert sufficient deodorizing effect. As a deodorizer to be used for a deodorizing fiber layer constituting a deodorizing filter, a chemisorption-type deodorizer, which can adsorb a malodorous component in a short time, exerts sufficient deodorizing effect during a pass through a deodorizing fiber layer, and has a high deodorizing speed and a large deodorizing capacity, is optimal. Meanwhile, there is no particular restriction on a form of a chemical bond in the chemisorption-type deodorizer, and it may depend occasionally on a functional group included in a chemisorption-type deodorizer, a functional group included in a malodorous component, or the like.

Specific examples of a malodorous component targeted by a chemisorption-type deodorizer include a basic compound, such as ammonia, and amine, an acidic compound, such as acetic acid, and isovaleric acid, an aldehyde, such as formaldehyde, acetaldehyde, and nonenal, and a sulfur compound, such as hydrogen sulfide, and methyl mercaptan.

Examples of a chemisorption-type deodorizer for the malodorous components include an inorganic chemisorption-type deodorizer and an organic chemisorption-type deodorizer. Specific examples of an inorganic chemisorption-type deodorizer include a phosphate of a tetravalent metal, zeolite, an amorphous complex oxide, a compound containing at least one kind of atom selected out of the group consisting of Ag, Cu, Zn, and Mn, a zirconium compound selected out of the group consisting of hydrated zirconium oxide and zirconium oxide, a hydrotalcite compound, and an amorphous active compound. Examples of an organic chemisorption-type deodorizer include an amine compound. As a deodorizer, which is superior in safety, and resistant to deterioration, an inorganic chemisorption-type deodorizer, which is insoluble or poorly soluble in water, is preferable.

The chemisorption-type deodorizers may be used solely or in a combination of 2 or more kinds. When a plurality of chemisorption-type deodorizers with respectively different deodorizing objects (malodorous components) are used, a synergistic effect may be obtained. For example, with respect to an excretion odor or putrid odor (odor of kitchen garbage, etc.) containing ammonia, trimethylamine, hydrogen sulfide, methyl mercaptan, dimethyl disulfide, etc. a combination of a chemisorption-type deodorizer for a basic gas and a chemisorption-type deodorizer for a sulfur-containing gas is appropriate; and for example, with respect to a body odor such as a sweat odor containing acetic acid, isovaleric acid, etc. a combination of a chemisorption-type deodorizer for a basic gas, and a chemisorption-type deodorizer for an acidic gas is appropriate. Further, with respect to a tobacco odor containing acetaldehyde, acetic acid, etc. a combination of a chemisorption-type deodorizer for a basic gas, a chemisorption-type deodorizer for an acidic gas, and chemisorption-type deodorizer for an aldehyde gas is suitable. When 2 or more kinds of chemisorption-type deodorizers are used in a combination, the ratio of the amounts to be used should preferably be selected taking into consideration the deodorizing performances such as deodorizing capacity and deodorizing speed of chemisorption-type deodorizers to be used, and a gas concentration in an objective environment (concentration of a malodorous component) in an environment. For example, when a malodorous gas containing a plurality of malodorous components is deodorized using 2 kinds of chemisorption-type deodorizers, their approximate mass ratio is from 20:80 to 80:20 for obtaining a sufficient deodorizing effect. The chemisorption-type deodorizer of the invention may be used together with a physical adsorption-type deodorizer such as activated carbon. In this regard, “deodorizing capacity” means an amount under standard conditions (mL) of a malodorous component, which 1 g of a chemisorption-type deodorizer is able to deodorize, and when the value is larger, longer durability of a deodorizing effect of a deodorizing filter may be obtained.

A deodorizing filter for a mask of the invention comprises 2 or more layers of the deodorizing fiber layers, and a chemisorption-type deodorizer contained in the respective deodorizing fiber layers may be the same of different. Chemisorption-type deodorizers contained in the respective deodorizing fiber layers are preferably the same from the viewpoint of deodorizing performance.

Next, a chemisorption-type deodorizer to be used of the invention will be described.

[1] Phosphate of Tetravalent Metal

A phosphate of a tetravalent metal is preferably a compound expressed by the following general formula (1). The compound is insoluble or poorly soluble in water, and is superior in a deodorizing effect on a basic gas.

HₐM_{b}(PO₄)_{c}.nH₂O (1)

(In formura, M is a tetravalent metal atom, a, b, and c are integers satisfying the equation a+4b=3c, and n is 0 or a positive integer.)

Examples of M in Formula (1) include Zr, Hf, Ti, and Sn.

Preferable specific examples of a phosphate of a tetravalent metal include zirconium phosphate (Zr(HPO₄)₂.H₂0), hafnium phosphate, titanium phosphate, and tin phosphate. Although they may be crystalline with various crystal systems, such as a-type crystal, (3-type crystal, and y-type crystal, or amorphous, either form may be used favorably.

[2] Amine Compound

An amine compound is preferably a hydrazine compound or an aminoguanidine salt. Since the compounds react with an aldehyde-containing gas, they are superior in a deodorizing effect on an aldehyde-containing gas. Examples of a hydrazine compound include adipic acid dihydrazide, carbohydrazide, succinic acid dihydrazide, and oxalic acid dihydrazide. Examples of an aminoguanidine salt include aminoguanidine hydrochloride, aminoguanidine sulfate, and aminoguanidine bicarbonate. The amine compounds are able to constitute a deodorizer supported on a carrier. In this case, a carrier is preferably an inorganic compound, and specific examples thereof include zeolite, an amorphous complex oxide, and silica gel as described below. Since both zeolite and an amorphous complex oxide have a deodorizing effect on a basic gas, when they are used as a carrier, they are effective on both an aldehyde-containing gas and a basic gas.

[3] Zeolite

Zeolite is preferably synthesis zeolite. Such zeolite is insoluble or poorly soluble in water, and superior in a deodorizing effect on a basic gas. There are various structures of zeolite, such as A-type, X-type, Y-type, α-type, β-type, ZSM-5, and amorphous type, and any known zeolite may be used.

[4] Amorphous Complex Oxide

An amorphous complex oxide is a compound other than the zeolite, and is preferably an amorphous complex oxide constituted with at least 2 kinds selected out of the group consisting of Al₂O₃, SiO₂, MgO, CaO, SrO, BaO, ZnO, ZrO₂, TiO₂, WO₂, CeO₂, Li₂O, Na₂O, and K₂O. The complex oxide is insoluble or poorly soluble in water and is superior in a deodorizing effect on a basic gas. An amorphous complex oxide expressed by X₂O—Al₂O₃—SiO₂ (X is at least one kind of alkali metal atom selected out of the group consisting of Na, K, and Li) is especially preferable, because it is superior in deodorizing performance. To be amorphous means that a clear diffraction signal based on a crystal face is not recognized in a powder X-ray diffraction analysis, and specifically that a leptokurtic (namely, sharp) signal peak scarcely appears in an X-ray diffraction chart, which plots a diffraction angle on the abscissa and a diffraction signal intensity on the ordinate.

[5] Complex Containing at Least One Kind of Atom Selected out of the Group Consisting of Ag, Cu, Zn, and Mn

The complex is insoluble or poorly soluble in water, and is superior in a deodorizing effect on a sulfur-containing gas. The complex is a composite material of at least one kind of atom selected out of the group consisting of Ag, Cu, Zn, and Mn, and at least one kind selected out of the group consisting of compounds including the atom(s) as well as another material. A compound including at least one kind of atom out of Ag, Cu, Zn, and Mn is preferably an oxide, a hydroxide, a salt of an inorganic acid, such as phosphoric acid, and sulfuric acid, and a salt of an organic acid, such as acetic acid, oxalic acid, and acrylic acid. Consequently, as a deodorizer [5] a water-insoluble complex, in which at least one kind of metal selected out of the group consisting of Ag, Cu, Zn, and Mn, or the above compound is supported on a carrier composed of an inorganic compound as another material, may be used. An inorganic compound preferable as a carrier is silica, a phosphate of a tetravalent metal, zeolite, or the like. In this regard, since a phosphate of a tetravalent metal, and zeolite have a deodorizing effect on a basic gas, when a tetravalent metal, or zeolite is used as a carrier, it is effective on both a sulfur-containing gas and a basic gas.

[6] Zirconium Compound

Examples of a zirconium compound include hydrated zirconium oxide, and zirconium oxide, and it is preferably an amorphous compound. Such compounds are insoluble or poorly soluble in water, and superior in a deodorizing effect on an acidic gas. Hydrated zirconium oxide is the same compound as zirconium oxyhydroxide, zirconium hydroxide, hydrous zirconium oxide, and zirconium oxide hydrate.

[7] Hydrotalcite Compound

A hydrotalcite compound has a hydrotalcite structure, and is preferably a compound expressed by the following general formula (2). The compound is insoluble or poorly soluble in water, and superior in a deodorizing effect on an acidic gas.

M¹₍₁₋ₓ₎M²ₓ(OH)₂Aⁿ⁻_{(x/n)}.mH₂O (2)

(In formula, M¹ is a divalent metal atom, M² is a trivalent metal atom, x is a number higher than 0 and not higher than 0.5, Aⁿ⁻ is an n-valent anion, such as a carbonate ion, and a sulfate ion, and m is a positive integer.)

Examples of such a hydrotalcite compound include magnesium-aluminum hydrotalcite, and zinc-aluminum hydrotalcite. Among them, magnesium-aluminum hydrotalcite is especially preferable, because the same has a superior deodorizing effect on an acidic gas. In this regard, a calcined product of hydrotalcite, namely a compound obtained by calcining a hydrotalcite compound at a temperature of about 500° C. or higher for eliminating a carbonate or a hydroxy group, is also included in a hydrotalcite compound.

[8] Amorphous Active Oxide

The amorphous active oxide is a compound not including the amorphous complex oxide, is preferably insoluble or poorly soluble in water, and is superior in a deodorizing effect on an acidic gas, or a sulfur-containing gas. Specific examples of the amorphous active oxide include Al₂O₃, SiO₂, MgO, CaO, SrO, BaO, ZnO, CuO, MnO, ZrO₂, TiO₂, WO₂, and CeO₂. Further, a surface-treated active oxide may be also used. Specific examples of a surface-treated product include an active oxide surface-treated with an organopolysiloxane, and an active oxide surface-coated with an oxide or a hydroxide of aluminum, silicon, zirconium, or tin. A surface treatment with an organic material such as an organopolysiloxane is more preferable than a surface treatment with an inorganic material, because the deodorizing performance is higher.

There is no particular restriction on the shape of a chemisorption-type deodorizer of the invention. With respect to the dimension of a chemisorption-type deodorizer, in a case in which it is a granule, the median diameter measured with a laser diffraction particle size distribution analyzer is preferably from 0.05 to 100 μm, more preferably from 0.1 to 50 μm, and yet more preferably from 0.2 to 30 μm from the viewpoint of deodorizing efficiency. When the dimension of a chemisorption-type deodorizer is in the range, a surface area to be exposed per unit mass of a chemisorption-type deodorizer becomes appropriate so that a sufficient deodorizing effect can be obtained, and when a desired basis weight is established, sufficient air permeability can be obtained.

The higher the efficiency of contact between a chemisorption-type deodorizer and a malodorous component is, the higher deodorizing effect can be obtained. Consequently, the specific surface area is preferably from 10 to 800 m²/g, and more preferably from 30 to 600 m²/g. A specific surface area can be measured by a BET method, which calculates the same from a nitrogen adsorption amount.

In a deodorizing fiber layer constituting a deodorizing filter for a mask of the invention, the content of a chemisorption-type deodorizer per unit is preferably as high as possible. Since, however, as the content increases, the air permeability of a deodorizing filter decreases and the cost increases, the content is ordinarily decided taking this into consideration. The content of a chemisorption-type deodorizer per 1 kind in a deodorizing fiber layer is preferably 1 g/m² or more, more preferably 3 g/m² or more, and yet more preferably 5 g/m² or more. Further, it is preferably 100 g/m² or less. When 2 or more kinds of chemisorption-type deodorizers are contained, the total content is preferably 2 g/m² or more, more preferably 6 g/m² or more, and yet more preferably 10 g/m² or more. Further, it is preferably 100 g/m² or less.

In a preferable aspect of a deodorizing fiber layer, with which a superior deodorizing effect is obtained of the invention, the content of a chemisorption-type deodorizer with respect to the mass of a fiber constituting the deodorizing fiber layer as 100 parts by mass is preferably from 2 to 60 parts by mass, more preferably from 5 to 55 parts by mass, and yet more preferably from 10 to 50 parts by mass.

The structure of a deodorizing fiber layer may be in the form of an aspect, in which a chemisorption-type deodorizer is buried in a surface of a fiber, or an aspect, in which a fiber and a chemisorption-type deodorizer ware bonded together with a binder (a binding agent) such as an emulsion. Examples of a binder in the latter case include a natural resin, a natural resin derivative, a phenol resin, a xylene resin, a urea resin, a melamine resin, a ketone resin, a coumarone-indene resin, a petroleum resin, a terpene resin, a cyclized rubber, a chlorinated rubber, an alkyd resin, a polyamide resin, a poly(vinyl chloride) resin, an acrylic resin, a vinyl chloride-vinyl acetate copolymer resin, a polyester resin, poly(vinyl acetate), poly(vinyl alcohol), poly(vinyl butyral), a chlorinated polypropylene, a styrene resin, an epoxy resin, a urethane resin, a cellulose derivative, starch, polyacrylamide, a poly(alkylene oxide), and poly(vinylpyrrolidone). Among them polyester, poly(vinyl alcohol), celluloses, starch, polyacrylamide, a poly(alkylene oxide), and poly(vinylpyrrolidone) are preferable, because a deodorizing mask produced with the deodorizing filter does not emit any unpleasant odor even after storage in a tightly closed environment, and polyester, poly(vinyl alcohol), and cellulose are more preferable. The polymers may be used solely or in a combination of 2 or more kinds.

As polyester, either of an aromatic polyester and an aliphatic polyester may be used, or they may be used in a combination. Further, the polyester may be either of a saturated polyester and an unsaturated polyester. As the polyester, a saturated polyester composed of a polycondensation product obtained using an acid component and a component containing a hydroxy group is preferable, and it may be a polyester bonded with a hydrophilic group, such as —SO₃H, —SO₃Na, —SO₃′, —COOH, —COO⁻, —OPO(OH)₂, and —OPO(OH)O⁻.

Examples of the acid component include terephthalic acid, isophthalic acid, orthophthalic acid, naphthalenedicarboxylic acid, 4,4′-diphenyldicarboxylic acid, trimellitic acid, trimesic acid, pyromellitic acid, benzoic acid, p-oxybenzoic acid, p-(hydroxyethoxy)benzoic acid, succinic acid, adipic acid, azelaic acid, sebacic acid, glutaric acid, suberic acid, dodecanedicarboxylic acid, fumaric acid, maleic acid, itaconic acid, 1,4-cyclohexanedicarboxylic acid, 1,3-cyclohexanedicarboxylic acid, cyclobutanetetracarboxylic acid, dimethylolpropionic acid, tricyclodecanedicarboxylic acid, tetrahydroterephthalic acid, tetrahydroorthophthalic acid, hexahydroorthophthalic acid, and a methyl ester or an anhydride of such di-, tri-, or tetra-carboxylic acid.

Examples of an acid component having a hydrophilic group include sulfonate compounds, such as sodium 5-sulfoisophthalic acid, ammonium 5-sulfoisophthalic acid, sodium 4-sulfoisophthalic acid, ammonium 4-methylsulfoisophthalic acid, sodium 2-sulfoterephthalic acid, potassium 5-sulfoisophthalic acid, potassium 4-sulfoisophthalic acid, and potassium 2-sulfoterephthalic acid.

Examples of the component including a hydroxy group include ethylene glycol, diethylene glycol, triethylene glycol, tetraethylene glycol, propylene glycol, dipropylene glycol, 1,3-propanediol, 2-methyl-1,3-propanediol, 1,4-butanediol, 1,2-butanediol, 1,5-pentanediol, 2-methyl-1,5-pentanediol, 1,6-hexanediol, neopentyl glycol, 2-ethyl-2-butyl-1,3-propanediol, 1,8-octanediol, 1,9-nonanediol, 1,10-decanediol, 2,2,4-trimethyl-1,3-pentanediol, polyethylene glycol, polypropylene glycol, poly(tetramethylene glycol), trimethylolpropane, trimethylolethane, glycerine, pentaerythritol, a bisphenol-ethylene oxide adduct, a bisphenol-propylene oxide adduct, 1,4-cyclohexanedimethanol, 1,4-cyclohexanediol, 1,3-cyclohexanedimethanol, 1,3-cyclohexanediol, hydrogenated bisphenol A, spiroglycol, tricyclodecanediol, tricyclodecanedimethanol, resorcinol, and 1,3-bis(2-hydroxyethoxy)benzene.

The polyester may be one obtained by a known process, such as a melt polymerization process, a solution polymerization process, and a solid phase polymerization process.

A hydrophilic group may be introduced by a known method, and when —COO⁻ is introduced, for example, a method, by which after a polycondensation reaction using anhydrous trimellitic acid, trimellitic acid, anhydrous pyromellitic acid, pyromellitic acid, trimesic acid, cyclobutanetetracarboxylic acid, dimethylolpropionic acid, etc., a neutralization reaction is performed using an amino compound, ammonia, or an alkali metal salt, is applied.

The poly(vinyl alcohol) is ordinarily a resin obtained using a vinyl ester, such as vinyl formate, vinyl acetate, vinyl propionate, and vinyl pivalate, and, for example, a resin obtained by the following method (A) or (B), and further a resin having a primary to tertiary amino group, or a quaternary ammonium group in the main chain or a side chain of poly(vinyl alcohol) may be used.
(A) Poly(vinyl alcohol) obtained by polymerizing a vinyl ester, and then saponifying the polymer, and
(B) Poly(vinyl alcohol) obtained by copolymerizing a vinyl ester and an ethylenic unsaturated monomer, and then saponifying the copolymer.

Examples of an ethylenic unsaturated monomer usable in the method (B) include an a-olefin, such as ethylene, propylene, isopropylene, butylene, isobutylene, pentylene, hexylene, cyclohexylene, cyclohexylethylene, and cyclohexylpropylene; acrylic acid, methacrylic acid, fumaric acid (anhydride), maleic acid (anhydride), itaconic acid (anhydride), acrylonitrile, methacrylonitrile, acrylamide, methacrylami de, trimethyl(3-acrylamide-3-dimethylpropyl)ammonium chloride, acrylamide-2-methylpropanesulfonic acid and a sodium salt thereof, ethyl vinyl ether, butyl vinyl ether, N-vinylpyrrolidone, vinyl chloride, vinyl bromide, vinyl fluoride, vinylidene chloride, vinylidene fluoride, tetrafluoroethylene, sodium vinyl sulfonate, and sodium allyl sulfonate.

Examples of celluloses include ethylcellulose, cellulose acetate propionate, cellulose acetate butyrate, methylcellulose, cellulose acetate, and cellulose butyrate.

Examples of starch include a modified starch, such as oxidized starch, etherified starch, and esterified starch.

As polyacrylamide, a product obtained by copolymerization of acrylamide (or methacrylamide); at least one kind selected out of a cationic monomer, and an anionic monomer; and another monomer such as a cross-linking agent, may be used.

Examples of a poly(alkylene oxide) include poly(ethylene oxide), poly(propylene oxide), an ethylene oxide-propylene oxide copolymer, a product obtained by reacting the poly(alkylene oxide) with a multivalent carboxylic acid, or an anhydride thereof, or a lower alkyl ester thereof, and a product obtained by reacting the poly(alkylene oxide) with diisocyanate.

Examples of a poly(vinylpyrrolidone) include a homopolymer of a vinylpyrrolidone, such as N-vinyl-2-pyrrolidone, and N-vinyl-4-pyrrolidone (namely poly(vinylpyrrolidone)), and a copolymer obtained by using a vinylpyrrolidone and a vinyl monomer.

Examples of the vinyl monomer include a fatty acid vinyl ester, such as vinyl acetate, vinyl propionate, and vinyl lactate; a vinyl ether, such as cyclohexyl vinyl ether, ethyl vinyl ether, hydroxyethyl vinyl ether, hydroxybutyl vinyl ether, and hydroxycyclohexyl vinyl ether; an acrylic acid ester or methacrylic acid ester, such as methyl acrylate, methyl methacrylate, ethyl acrylate, ethyl methacrylate, 2-hydroxyethyl acrylate, 2-hydroxyethyl methacrylate, 2-hydroxypropyl acrylate, 2-hydroxypropyl methacrylate, 3-hydroxypropyl acrylate, and 3-hydroxypropyl methacrylate; and an allyl ether, such as hydroxybutyl allyl ether, and ethylene glycol monoallyl ether.

When a processing is conducted using a deodorizer-containing processing liquid, in which a chemisorption-type deodorizer and a binder are combined, there is no particular restriction on the relative content of a chemisorption-type deodorizer with respect to a resin solid content originated from an emulsion in a deodorizer-containing processing liquid. However, a higher binder content is preferable from a viewpoint that the binding strength of a deodorizer is enhanced and falling off of a deodorizer is mitigated. On the other hand, when the content of a binder resin solid content is lower, a deodorizer contacts a malodorous gas easier, and therefore a deodorizing effect becomes higher. Therefore, as a good balance between the two, the respective contents of a binder (solid content) and a chemisorption-type deodorizer are preferably in the ranges of from 10 to 90 mass % and from 10 to 90 mass % with respect to the total of a binder (solid content) and a deodorizer as 100 mass %, and more preferably in the ranges of from 25 to 60 mass % and from 40 to 75 mass %.

When an additive as a binder is added to a deodorizer-containing processing liquid containing a chemisorption-type deodorizer, impartation of multifunctions in addition to deodorizing performance, or improvement processability can be attained. Examples of the additive include a dispersing agent, an antifoaming agent, a viscosity regulator, a pigment, a dye, an aromatic agent, a physical adsorption-type deodorizer, an antibacterial agent, an antiviral agent, and an anti-allergen agent. As for the amount of an additive, an appropriate amount should be decided considering the purpose, and it is required that the deodorizing effect of a chemisorption-type deodorizer should not be impaired, and the physical properties and mask processability of a deodorizing nonwoven fabric should not be affected.

As a preparation method for a deodorizer-containing processing liquid containing a chemisorption-type deodorizer and a binder, a general dispersing method for an inorganic powder may be applied. For example, after adding an additive such as a dispersing agent into an emulsion of a polyester resin, and further a chemisorption-type deodorizer, the mixture may be dispersed with stirring by a sand mill, a disper, a ball mill, or the like. When the solid content concentration of a chemisorption-type deodorizer in a composition containing a deodorizer is higher, the viscosity of a deodorizer-containing processing liquid increases and the handling becomes more difficult that much, meanwhile the stability tends to be improved. Therefore, the solid content concentration of a deodorizer in a deodorizer-containing processing liquid is preferably from 5 to 60 mass %. A viscosity regulator, etc. may be added for regulating the viscosity of a deodorizer-containing processing liquid to the extent that the deodorizing performance is not affected.

When the amount of a chemisorption-type deodorizer per unit area of a deodorizing fiber layer in a deodorizing filter produced using the deodorizer-containing processing liquid is increased in order to improve a deodorizing effect, in general the used amount of a binder for binding a chemisorption-type deodorizer increases also, such that the binder is deposited among fibers constituting a deodorizing fiber layer and the air-permeability of a deodorizing filter is decreased. Further, the amount of a chemisorption-type deodorizer to be buried in a binder increases such that contact with a malodorous component contained in a malodorous gas is inhibited and a deodorizing effect expected from an increase in the content of a deodorizer cannot be obtained. Therefore for developing the deodorizing effect of a chemisorption-type deodorizer thoroughly without decreasing the air-permeability, the thickness and basis weight of each deodorizing fiber layer is required to be in a specific range in a deodorizing filter of the invention.

The thickness of a deodorizing fiber layer in a deodorizing filter for a mask of the invention is from 0.15 to 0.4 mm, preferably from 0.18 to 0.38 mm, and more preferably from 0.2 to 0.35 mm. When the thickness of a deodorizing fiber layer is less than 0.15 mm, the deodorizing performance may occasionally decrease. Meanwhile, when the thickness exceeds 0.4 mm, the air permeability of a deodorizing filter decreases.

The basis weight (mass per 1m²) of a deodorizing fiber layer is from 20 to 45 g/m², preferably from 22 to 42 g/m², and more preferably from 25 to 40 g/m² because a sufficient deodorizing effect and air-permeability are obtained. When the basis weight of a deodorizing fiber layer is less than 20 g/m², the air permeability of a deodorizing fiber layer becomes too high such that a malodorous component in a malodorous gas does not contact a chemisorption-type deodorizer, and most of a malodorous gas passes a deodorizing fiber layer intact, and therefore the deodorizing effect decreases. Meanwhile, when the basis weight exceeds 45 g/m², the air permeability of a deodorizing fiber layer decreases remarkably such that a gas does not flow smoothly from one side of a deodorizing filter to the other side.

When the thickness of a deodorizing fiber layer is from 0.15 to 0.4 mm, and the basis weight is from 20 to 45 g/m², a high air-permeability is secured, while a malodorous component is adsorbed sufficiently by a chemisorption-type deodorizer, so that superior deodorizing performance with respect to a malodorous gas can be obtained. For a deodorizing filter to have high air-permeability and to develop high deodorizing performance, it is important that the thickness and the basis weight of a deodorizing fiber layer should be in specific ranges.

Further, by dividing a specific deodorizing nonwoven fabric layer from monolayer to 2 layers, a chemisorption-type deodorizer is exposed more easily to a nonwoven fabric surface, so as to improve the deodorizing performance. Further, by providing a deodorizing nonwoven fabric layer with 2 or more layers, the processed amount of a chemisorption-type deodorizer per a layer is decreased, so that the applicability onto a nonwoven fabric is improved and a deodorizer can be applied to a nonwoven fabric uniformly, and higher deodorizing performance can be attained.

A deodorizing filter for a mask of the invention is preferably provided with 2 to 8 layers of deodorizing fiber layers, more preferably 2 to 4 layers, yet more preferably 2 or 3 layers, and especially preferably 2 layers.

The air-permeability of the deodorizing filter for a mask comprising 2 or more layers of deodorizing fiber layers is preferably from 50 to 350 cm³/(cm²·s) in terms of air permeability according to a Frazier type method, more preferably from 60 to 300 cm³/(cm²·s), and yet more preferably from 80 to 250 cm³/(cm²·s). When a filter with an air permeability according to a Frazier type method within the range of from 50 to 350 cm³/(cm²·s) is used for a mask, breathing is easy and a deodorizing effect can be well exhibited.

A deodorizing filter for a mask of the invention may be produced by any of various methods, and examples thereof include the following methods.
(1) A method for producing a deodorizing filter for a mask by preparing a deodorizing fiber layer by coating (dipping, spraying, padding, etc.) a deodorizer-containing processing liquid containing a chemisorption-type deodorizer and a binder on the entire woven fabric or nonwoven fabric composed of a fiber not containing a chemisorption-type deodorizer, followed by drying such that a chemisorption-type deodorizer is bound on a surface of a fiber composing a woven fabric or nonwoven fabric, and laminating 2 or more layers of the deodorizing fiber layer.
(2) A method for producing a deodorizing filter for a mask by preparing a deodorizing fiber layer by using a woven fabric or nonwoven fabric composed of a conjugated fiber burying a chemisorption-type deodorizer in a substrate surface of the fiber such that it is exposed outward, and, if necessary, applying an entanglement treatment (needle punch method, etc.), and laminating 2 or more layers of the deodorizing fiber layer.
(3) A method for producing a deodorizing filter for a mask by preparing a deodorizing fiber layer by applying a heat treatment or a chemical treatment to a fiber of a woven fabric or a nonwoven fabric composed of a fiber not containing a chemisorption-type deodorizer, in a state where the fiber is in contact with a chemisorption-type deodorizer, such that the chemisorption-type deodorizer is fixed on to a fiber surface, and laminating 2 or more layers of the deodorizing fiber layer.

Of the invention, a spreading process method in (1) is preferable, and a method for producing a deodorizing filter for a mask by preparing a deodorizing fiber layer by dipping a fiber in a deodorizer-containing processing liquid containing a chemisorption-type deodorizer and a binder, and laminating 2 or more layers of the deodorizing fiber layer, is more preferable.

2. Deodorizing Mask

A deodorizing mask of the invention is a mask comprising 2 or more layers of the deodorizing fiber layer (deodorizing nonwoven fabric layer) containing a fiber and a chemisorption-type deodorizer. A deodorizing mask of the invention is preferably provided with, in addition to a deodorizing nonwoven fabric layer, a dust-tight nonwoven fabric layer, and another nonwoven fabric layer, wherein the dust-tight nonwoven fabric layer is positioned on the facial side. A dust-tight nonwoven fabric layer and another nonwoven fabric layer may be constituted with a laminate of a plurality of nonwoven fabrics. With such a constitution, a superior deodorizing property is obtained and inhalation of a malodorous gas can be suppressed.

Further, in a deodorizing mask of the invention another layer may be placed between a deodorizing nonwoven fabric layer and a dust-tight nonwoven fabric layer to the extent that the effect of the invention be not affected. There is no particular restriction on the structure, such as shape and a material, of such other layer, insofar as it has air-permeability, and either of a nonwoven fabric layer and a woven fabric layer is acceptable. Preferably it has an air-permeability not lower than a deodorizing nonwoven fabric layer.

A deodorizing mask of the invention should preferably have a structure in which a deodorizing nonwoven fabric layer is adjacent to a dust-tight nonwoven fabric layer. When a deodorizing nonwoven fabric layer is placed in contact with a dust-tight nonwoven fabric layer, the effect of the invention may be exhibited efficiently.

In producing a deodorizing mask of the invention, it is preferable not to bond a deodorizing filter for a mask of the invention and a dust protecting nonwoven fabric together at a breathing part (ordinarily, a part surrounded by rims), but to bond them only at rims. In other words, they may be fixed for preventing a nonwoven fabric constituted with a multilayered body from displacing internally, at rims of a mask main part excluding a breathing part by means of heat sealing, gluing, sewing, or the like. Other nonwoven fabrics may be further placed on the facial side and the open air side. Although there is no particular restriction on the resin type, etc. of such other nonwoven fabric, it should preferably have an air-permeability as high as, and more preferably twice or more as high as both of a deodorizing nonwoven fabric and a dust protecting nonwoven fabric. For example, on the open air side a water repellent nonwoven fabric such as a polypropylene-made nonwoven fabric is favorably used, and on the facial side a flexible rayon-made or polyolefin-made nonwoven fabric is preferably used.

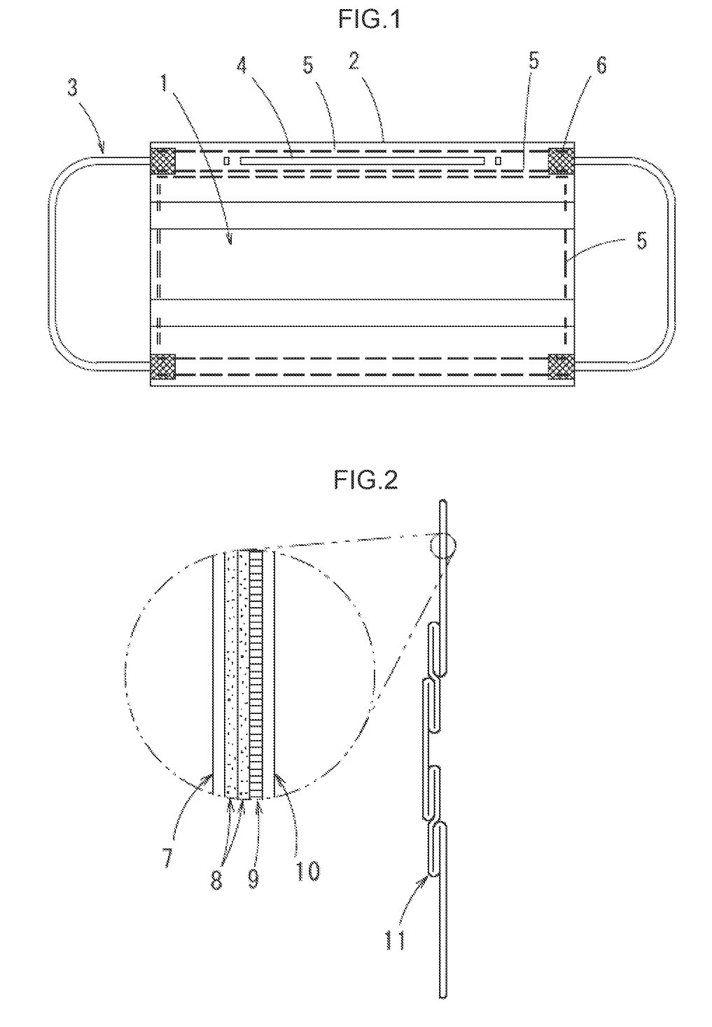

Except selection of a nonwoven fabric constituting a mask main part and a laminating method, a production method itself for a spatial structure mask with such a shape has been known to a person skilled in the art. As for the shape of a mask main part, based on the rectangular shape as shown in FIG. 1 in an approximate size of 10 cm×18 cm, the shape or size of a pleat, etc. may be decided appropriately. As for parts, such as a nose wire (a wire or a resin for retaining the shape of a part of rim of a mask main part to conform to the shape of nose), an ear loop, and a reinforcement sticker, those known may be used appropriately. Further, a heat sealer, etc. for assembly during production, a conventional device may be used.

Meanwhile, FIG. 2 is a schematic sectional view of an example of a deodorizing mask of the invention, in which an open air side polypropylene (PP) nonwoven fabric layer 7, two layers of deodorizing nonwoven fabric layers (deodorizing fiber layers) 8, a dust-tight nonwoven fabric layer 9, and a facial side PP nonwoven fabric layer 10 are layered one on another in the mentioned order from the open air side of a deodorizing mask to the facial side. Further, in the deodorizing mask shown in FIG. 2, pleats 11 are provided.

### EXAMPLES

The present invention will be described by way of Examples below, provided that the invention be not limited thereto. Meanwhile, the expressions of “part” and “%” herein are by mass, unless otherwise specified.

1. Base Material for Deodorizing Mask

A deodorizing mask was produced as an omega pleated mask using a deodorizing nonwoven fabric prepared by using a base material constituted with the following nonwoven fabric sheet, and a deodorizer-containing processing liquid containing a deodorizer shown in Table 1, a polyester binder resin, and water, and another nonwoven fabric. The above will be described in details below.

### (Nonwoven Fabric Sheet W1)

A nonwoven fabric comprising a polypropylene resin fiber and a polyethylene resin fiber at a mass ratio of 1:1 produced by a thermal bonding method. The basis weight was 20 g/m².

### (Nonwoven Fabric Sheet W2)

A nonwoven fabric constituted with a polyester resin fiber produced by a thermal bonding method. The basis weight was 17 g/m².

### (Nonwoven Fabric Sheet W3)

A nonwoven fabric comprising a polypropylene resin fiber and a polyethylene resin fiber at a mass ratio of 1:1 produced by a thermal bonding method. The basis weight was 40 g/m².

TABLE 1

| | | | Average |
|---|---|---|---|
| | | Deodorizing | particle |
| | | capacity | diameter |
| Deodorizer | Objective odor | (mL/g) | (μm) |
| Zirconium phosphate | Ammonia | 150 | 0.8 |
| Aluminum silicate | Ammonia | 34 | 12 |
| CuO•SiO₂ complex oxide | Methyl mercaptan | 50 | 3 |
| Active zinc oxide | Acetic acid | 28 | 14 |
| Hydrous zirconium oxide | Acetic acid | 32 | 1 |
| Hydrotalcite | Acetic acid | 48 | 5 |
| Adipic acid dihydrazide | Acetaldehyde | 38 | 5 |
| (30%)-supported silica gel | | | |
| Amorphous zeolite | Ammonia | 53 | 4 |
| Activated carbon | Ammonia | 10 | 3 |

An average particle diameter of a deodorizer shown in Table 1 is a median diameter measured with a laser diffraction particle size distribution analyzer on a volume basis.

A test method for calculating the deodorizing capacity of a deodorizer is as follows.

Into a Tedlar bag with a capacity of approx. 4 L, 0.01 g of a deodorizer was placed, the bag was closed tightly, and thereafter 2 L of a gas containing ammonia (8000 ppm), methyl mercaptan (40 ppm), acetic acid (380 ppm), or acetaldehyde (2000 ppm) corresponding to 200 times a concentration of odor intensity 5 was injected. After 24 hours the concentration of each malodorous component (residual concentration of gas component) was measured with a gas detector tube, and a deodorizing capacity (mL/ g) was determined according to the following equation.

Deodorizing capacity (mL/ g)=[2000 (mL)×(initial concentration of malodorous gas component (ppm)−residual concentration of gas component (ppm))×101/0.01 (g)

2. Evaluation Method

### (1) Air Permeability

Air permeability of a deodorizing filter for a mask and a deodorizing mask using the same was measured by a Frazier type method according to JIS L1096 “Testing methods for woven and knitted fabrics” (revised in 2010). The unit is cm³/(cm²·s).

### (2) Evaluation of Deodorizing Mask

### (a) Measurement of Reduction Rate of Malodorous Component

A malodorous gas prepared in advance to contain a malodorous component at a predetermined concentration was passed through the main part of a deodorizing mask form one side to the other side as a deodorizing test. Specifically, a malodorous gas contained in a bag was aspirated by a gas sampler “Model GV-100” (Model type) produced by Gastec Corporation trough a deodorizing mask with an area of 5 cm² located in the pathway, and then the concentration of a malodorous component in a passing gas was measured with a gas detector tube.

As a malodorous gas, a gas containing ammonia (200 ppm), acetic acid (9.5 ppm), or acetaldehyde (50 ppm) corresponding to 5 times a concentration of odor intensity 5 based on Six Grades Odor Intensity Measurement Method, as well as a gas containing methyl mercaptan (10 ppm) corresponding to 50 times a concentration of odor intensity 5 were passed. After passing, the concentration of each malodorous component in a passing gas was measured using a gas detector tube corresponding to each malodorous component (gas detector tube for ammonia: No. 3M, gas detector tube for acetic acid: No. 81L, gas detector tube for acetaldehyde: No. 92, and gas detector tube for methyl mercaptan: No. 71), and a malodorous component reduction rate was determined according to the following equation.

Malodorous component reduction rate=[(concentration of malodorous component before gas flow−concentration of malodorous component after gas flow)/concentration of malodorous component before gas flow]×100

### (b) Sensory Test Wearing Mask

An ammonia-containing gas (40 ppm), an acetic acid-containing gas (1.9 ppm), an acetaldehyde-containing gas (10 ppm), or a methyl mercaptan-containing gas (0.2 ppm) equivalent to the concentration of odor intensity 5 in an amount of 2 L was filled in an odor gas bag, and 6 test subjects were made to sniff the odor in the odor gas bag to identify the odor of a malodorous gas, and then each of the 6 subjects was made to judge an odor intensity according to the following criteria by sniffing the odor in the odor gas bag wearing a deodorizing mask. Odor intensities by the 6 subjects were averaged, and determined as an odor intensity by a sensory test. A lower value of odor intensity means a higher deodorizing effect of a mask.

Odor intensity 0: odorless

Odor intensity 1: perceivable odor

Odor intensity 2: weak but barely discernible odor

Odor intensity 3: easily discernible odor

Odor intensity 4: rather strong odor

Odor intensity 5: very strong odor

### (c) Hedonic Scale

In a sampling bag, 10 deodorizing masks were placed, and the bag was closed tightly, to which 5 L of odorless air was added, and then stored in a thermostatic chamber at 50° C. for 30 days. Thereafter, 6 test subjects were made to wear a mask, breathe 5 times through the nose, and sniff the odor thereof. The quality of an odor was judged according to the criteria in Table 2, and an average of values by 6 subjects was determined as the result of a hedonic scale evaluation.

TABLE 2

| Quality of odor | Hedonic scale |
|---|---|
| Deeply uncomfortable | −4 |
| Very uncomfortable | −3 |
| Uncomfortable | −2 |
| Slightly uncomfortable | −1 |
| Neutral | 0 |
| Slightly comfortable | 1 |
| Comfortable | 2 |
| Very comfortable | 3 |
| Extremely comfortable | 4 |

### 3. Production of Deodorizing Nonwoven Fabric

A deodorizing nonwoven fabric to be used as a deodorizing fiber layer was produced using a base material constituted with the above nonwoven fabric sheet, and a deodorizer-containing processing liquid containing a deodorizer shown in Table 1, a polyester-base binder, and water.

### Production Example 1

### Production of Deodorizing Nonwoven Fabric D1

A deodorizer-containing processing liquid with a solid content concentration of 10% was prepared using a zirconium phosphate powder, a CuO.SiO₂ complex oxide powder, and a polyester binder, at a mass ratio of 4 parts of zirconium phosphate, 4 parts of a CuO.SiO₂ complex oxide, and 4 parts of a polyester resin solid content. Next, the deodorizer-containing processing liquid was coated uniformly on nonwoven fabric sheet W1 by padding coating, such that the coating amount of zirconium phosphate became 4 g/m², and the coating amount of a CuO.SiO₂ complex oxide became 4 g/m², followed by drying at 130° C. to produce a deodorizing nonwoven fabric D1, in which a deodorizer was bound uniformly to nonwoven fabric sheet W1 entirely from one side to the other side. With respect thereto, the basis weight and the thickness as well as the air permeability for a single layer and a 2-layer laminate were measured (see Table 3).

### Production Example 2

### Production of Deodorizing Nonwoven Fabric D2

A deodorizer-containing processing liquid with a solid content concentration of 10% was prepared using a zirconium phosphate powder, a CuO.SiO₂ complex oxide powder, and a polyester binder, at a mass ratio of 3 parts of zirconium phosphate, 3 parts of a CuO.SiO₂ complex oxide, and 3 parts of a polyester resin solid content. Next, the deodorizer-containing processing liquid was coated uniformly on nonwoven fabric sheet W1 by padding coating, such that the coating amount of zirconium phosphate became 3 g/m², and the coating amount of a CuO.SiO₂ complex oxide became 3 g/m², followed by drying at 130° C. to produce a deodorizing nonwoven fabric D2, in which a deodorizer was bound uniformly to nonwoven fabric sheet W1 entirely from one side to the other side. With respect thereto, the basis weight and the thickness as well as the air permeability for a single layer and a 2-layer laminate were measured (see Table 3).

### Production Example 3

### Production of Deodorizing Nonwoven Fabric D3

A deodorizer-containing processing liquid with a solid content concentration of 10% was prepared using an aluminum silicate powder, a hydrous zirconium oxide powder, and a polyester binder, at a mass ratio of 4 parts of aluminum silicate, 3 parts of hydrous zirconium oxide, and 4 parts of a polyester resin solid content. Next, the deodorizer-containing processing liquid was coated uniformly on nonwoven fabric sheet W1 by padding coating, such that the coating amount of aluminum silicate became 4 g/m², and the coating amount of hydrous zirconium oxide became 3 g/m², followed by drying at 130° C. to produce a deodorizing nonwoven fabric D3, in which a deodorizer was bound uniformly to nonwoven fabric sheet W1 entirely from one side to the other side. With respect thereto, the basis weight and the thickness as well as the air permeability for a single layer and a 2-layer laminate were measured (see Table 3).

### Production Example 4

### Production of Deodorizing Nonwoven Fabric D4

A deodorizer-containing processing liquid with a solid content concentration of 10% was prepared using a zirconium phosphate powder, a CuO.SiO₂ complex oxide powder, Adipic acid dihydrazide (30%)-supported silica gel powder, and a polyester binder, at a mass ratio of 4 parts of zirconium phosphate, 4 parts of a CuO.SiO₂ complex oxide, 3 parts of Adipic acid dihydrazide (30%)-supported silica gel, and 6 parts of a polyester resin solid content. Next, the deodorizer-containing processing liquid was coated uniformly on nonwoven fabric sheet W1 by padding coating, such that the coating amount of zirconium phosphate became 4 g/m², the coating amount of a CuO.SiO₂ complex oxide became 4 g/m², and the coating amount of Adipic acid dihydrazide (30%)-supported silica gel became 3 g/m², followed by drying at 130° C. to produce a deodorizing nonwoven fabric D4, in which a deodorizer was bound uniformly to nonwoven fabric sheet W1 entirely from one side to the other side. With respect thereto, the basis weight and the thickness as well as the air permeability for a single layer and a 2-layer laminate were measured (see Table 3).

### Production Example 5

### Production of Deodorizing Nonwoven Fabric D5

A deodorizer-containing processing liquid with a solid content concentration of 10% was prepared using an aluminum silicate powder, an active zinc oxide powder, and a polyester binder, at a mass ratio of 4 parts of aluminum silicate, 3 parts of an active zinc oxide, and 4 parts of a polyester resin solid content. Next, the deodorizer-containing processing liquid was coated uniformly on nonwoven fabric sheet W1 by dip coating, such that the coating amount of aluminum silicate became 4 g/m², and the coating amount of an active zinc oxide became 3 g/m², followed by drying at 130° C. to produce a deodorizing nonwoven fabric D5, in which a deodorizer was bound uniformly to nonwoven fabric sheet W1 entirely from one side to the other side. With respect thereto, the basis weight and the thickness as well as the air permeability for a single layer and a 2-layer laminate were measured (see Table 3).

### Production Example 6

### Production of Deodorizing Nonwoven Fabric D6

A deodorizer-containing processing liquid with a solid content concentration of 10% was prepared using a hydrous zirconium oxide powder, Adipic acid dihydrazide (30%)-supported silica gel powder, and a polyester binder, at a mass ratio of 3 parts of hydrous zirconium oxide, 3 parts of Adipic acid dihydrazide (30%)-supported silica gel, and 3 parts of a polyester resin solid content. Next, the deodorizer-containing processing liquid was coated uniformly on nonwoven fabric sheet W1 by padding coating, such that the coating amount of hydrous zirconium oxide became 3 g/m², and the coating amount of Adipic acid dihydrazide (30%)-supported silica gel became 3 g/m², followed by drying at 130° C. to produce a deodorizing nonwoven fabric D6, in which a deodorizer was bound uniformly to nonwoven fabric sheet W1 entirely from one side to the other side. With respect thereto, the basis weight and the thickness as well as the air permeability for a single layer and a 2-layer laminate were measured (see Table 3).

### Production Example 7

### Production of Deodorizing Nonwoven Fabric D7

A deodorizer-containing processing liquid with a solid content concentration of 10% was prepared using an amorphous zeolite powder, a hydrotalcite powder, and a polyester binder, at a mass ratio of 4 parts of amorphous zeolite, 3 parts of hydrotalcite, and 4 parts of a polyester resin solid content. Next, the deodorizer-containing processing liquid was coated uniformly on nonwoven fabric sheet W1 by padding coating, such that the coating amount of amorphous zeolite became 4 g/m², and the coating amount of hydrotalcite became 3 g/m², followed by drying at 130° C. to produce a deodorizing nonwoven fabric D7, in which a deodorizer was bound uniformly to nonwoven fabric sheet W1 entirely from one side to the other side. With respect thereto, the basis weight and the thickness as well as the air permeability for a single layer and a 2-layer laminate were measured (see Table 3).

### Production Example 8

### Production of Deodorizing Nonwoven Fabric D8

A deodorizer-containing processing liquid with a solid content concentration of 10% was prepared using a zirconium phosphate powder, a CuO.SiO₂ complex oxide powder, a hydrous zirconium oxide powder, and a polyester binder, at a mass ratio of 4 parts of zirconium phosphate, 4 parts of a CuO.SiO₂ complex oxide, 3 parts of hydrous zirconium oxide, and 6 parts of a polyester resin solid content. Next, the deodorizer-containing processing liquid was coated uniformly on nonwoven fabric sheet W1 by padding coating, such that zirconium phosphate became 4 g/m², the coating amount of a CuO.SiO₂ complex oxide became 4 g/m², and the coating amount of hydrous zirconium oxide became 3 g/m², followed by drying at 130° C. to produce a deodorizing nonwoven fabric D8, in which a deodorizer was bound uniformly to nonwoven fabric sheet W1 entirely from one side to the other side. With respect thereto, the basis weight and the thickness as well as the air permeability for a single layer and a 2-layer laminate were measured (see Table 3).

### Production Example 9

### Production of Deodorizing Nonwoven Fabric D9

A deodorizer-containing processing liquid with a solid content concentration of 10% was prepared using an aluminum silicate powder, an active zinc oxide powder, Adipic acid dihydrazide (30%)-supported silica gel powder, and a polyester binder, at a mass ratio of 4 parts of aluminum silicate, 3 parts of active zinc oxide, 3 parts of Adipic acid dihydrazide (30%)-supported silica gel, and 5 parts of a polyester resin solid content. Next, the deodorizer-containing processing liquid was coated uniformly on nonwoven fabric sheet W1 by padding coating, such that the coating amount of aluminum silicate became 4 g/m², the coating amount of active zinc oxide became 3 g/m², and the coating amount of Adipic acid dihydrazide (30%)-supported silica gel became 3 g/m², followed by drying at 130° C. to produce a deodorizing nonwoven fabric D9, in which a deodorizer was bound uniformly to nonwoven fabric sheet W1 entirely from one side to the other side. With respect thereto, the basis weight and the thickness as well as the air permeability for a single layer and a 2-layer laminate were measured (see Table 3).

### Production Example 10

### Production of Deodorizing Nonwoven Fabric D10

A deodorizer-containing processing liquid with a solid content concentration of 10% was prepared using a CuO.SiO₂ complex oxide powder, a hydrotalcite powder, and a polyester binder, at a mass ratio of 4 parts of a CuO.SiO₂ complex oxide, 3 parts of hydrotalcite, and 4 parts of a polyester resin solid content. Next, the deodorizer-containing processing liquid was coated uniformly on nonwoven fabric sheet W1 by padding coating, such that the coating amount of a CuO.SiO₂ complex oxide became 4 g/m², and the coating amount of hydrotalcite became 3 g/m², followed by drying at 130° C. to produce a deodorizing nonwoven fabric D10, in which a deodorizer was bound uniformly to nonwoven fabric sheet W1 entirely from one side to the other side. With respect thereto, the basis weight and the thickness as well as the air permeability for a single layer and a 2-layer laminate were measured (see Table 3).

### Production Example 11

### Production of Deodorizing Nonwoven Fabric D11

A deodorizer-containing processing liquid with a solid content concentration of 10% was prepared using a zirconium phosphate powder, a CuO.SiO₂ complex oxide powder, and a polyester binder, at a mass ratio of 4 parts of zirconium phosphate, 4 parts of a CuO.SiO₂ complex oxide, and 4 parts of a polyester resin solid content. Next, the deodorizer-containing processing liquid was coated uniformly on nonwoven fabric sheet W2 by padding coating, such that the coating amount of zirconium phosphate became 4 g/m², and the coating amount of a CuO.SiO₂ complex oxide became 4 g/m², followed by drying at 130° C. to produce a deodorizing nonwoven fabric D11, in which a deodorizer was bound uniformly to nonwoven fabric sheet W2 entirely from one side to the other side. With respect thereto, the basis weight and the thickness as well as the air permeability for a single layer and a 2-layer laminate were measured (see Table 4).

Production Example 12

### Production of Deodorizing Nonwoven Fabric D12

A deodorizer-containing processing liquid with a solid content concentration of 10% was prepared using a zirconium phosphate powder, a CuO.SiO₂ complex oxide powder, and a polyester binder, at a mass ratio of 8 parts of zirconium phosphate, 8 parts of a CuO.SiO₂ complex oxide, and 8 parts of a polyester resin solid content. Next, the deodorizer-containing processing liquid was coated uniformly on nonwoven fabric sheet W3 by padding coating, such that the coating amount of zirconium phosphate became 8 g/m², and the coating amount of a CuO.SiO₂ complex oxide became 8 g/m², followed by drying at 130° C. to produce a deodorizing nonwoven fabric D12, in which a deodorizer was bound uniformly to nonwoven fabric sheet W3 entirely from one side to the other side. With respect thereto, the basis weight, the thickness, and the air permeability were measured (see Table 4).

### Production Example 13

### Production of Deodorizing Nonwoven Fabric D13

A deodorizer-containing processing liquid with a solid content concentration of 10% was prepared using a zirconium phosphate powder, a CuO.SiO₂ complex oxide powder, and a polyester binder, at a mass ratio of 6 parts of zirconium phosphate, 6 parts of a CuO.SiO₂ complex oxide, and 6 parts of a polyester resin solid content. Next, the deodorizer-containing processing liquid was coated uniformly on nonwoven fabric sheet W3 by padding coating, such that the coating amount of zirconium phosphate became 6 g/m², and the coating amount of a CuO.SiO₂ complex oxide became 6 g/m², followed by drying at 130° C. to produce a deodorizing nonwoven fabric D13, in which a deodorizer was bound uniformly to nonwoven fabric sheet W3 entirely from one side to the other side. With respect thereto, the basis weight, the thickness, and the air permeability were measured (see Table 4).

### Production Example 14

### Production of Deodorizing Nonwoven Fabric D14

A deodorizer-containing processing liquid with a solid content concentration of 10% was prepared using an aluminum silicate powder, a hydrous zirconium oxide powder, and a polyester binder, at a mass ratio of 8 parts of aluminum silicate, 6 parts of hydrous zirconium oxide, and 7 parts of a polyester resin solid content. Next, the deodorizer-containing processing liquid was coated uniformly on nonwoven fabric sheet W3 by padding coating, such that the coating amount of aluminum silicate became 8 g/m², and the coating amount of hydrous zirconium oxide became 6 g/m², followed by drying at 130° C. to produce a deodorizing nonwoven fabric D14, in which a deodorizer was bound uniformly to nonwoven fabric sheet W3 entirely from one side to the other side. With respect thereto, the basis weight, the thickness, and the air permeability were measured (see Table 4).

### Production Example 15

### Production of Deodorizing Nonwoven Fabric D15

A deodorizer-containing processing liquid with a solid content concentration of 10% was prepared using an aluminum silicate powder, an active zinc oxide powder, and a polyester binder, at a mass ratio of 8 parts of aluminum silicate, 6 parts of an active zinc oxide, and 7 parts of a polyester resin solid content. Next, the deodorizer-containing processing liquid was coated uniformly on nonwoven fabric sheet W3 by dip coating, such that the coating amount of aluminum silicate became 8 g/m², and the coating amount of an active zinc oxide became 6 g/m², followed by drying at 130° C. to produce a deodorizing nonwoven fabric D15, in which a deodorizer was bound uniformly to nonwoven fabric sheet W3 entirely from one side to the other side. With respect thereto, the basis weight, the thickness, and the air permeability were measured (see Table 4).

### Production Example 16

### Production of Deodorizing Nonwoven Fabric D16

A deodorizer-containing processing liquid with a solid content concentration of 10% was prepared using a hydrous zirconium oxide powder, Adipic acid dihydrazide (30%)-supported silica gel powder, and a polyester binder, at a mass ratio of 6 parts of hydrous zirconium oxide, 6 parts of Adipic acid dihydrazide (30%)-supported silica gel, and 6 parts of a polyester resin solid content. Next, the deodorizer-containing processing liquid was coated uniformly on nonwoven fabric sheet W3 by padding coating, such that the coating amount of hydrous zirconium oxide became 6 g/m², and the coating amount of Adipic acid dihydrazide (30%)-supported silica gel became 6 g/m², followed by drying at 130° C. to produce a deodorizing nonwoven fabric D16, in which a deodorizer was bound uniformly to nonwoven fabric sheet W3 entirely from one side to the other side. With respect thereto, the basis weight, the thickness, and the air permeability were measured (see Table 4).

### Production Example 17

### Production of Deodorizing Nonwoven Fabric D17

A deodorizer-containing processing liquid with a solid content concentration of 10% was prepared using an amorphous zeolite powder, a hydrotalcite powder, and a polyester binder, at a mass ratio of 8 parts of amorphous zeolite, 6 parts of hydrotalcite, and 7 parts of a polyester resin solid content. Next, the deodorizer-containing processing liquid was coated uniformly on nonwoven fabric sheet W3 by padding coating, such that the coating amount of amorphous zeolite became 8 g/m², and the coating amount of hydrotalcite became 6 g/m², followed by drying at 130° C. to produce a deodorizing nonwoven fabric D17, in which a deodorizer was bound uniformly to nonwoven fabric sheet W3 entirely from one side to the other side. With respect thereto, the basis weight, the thickness, and the air permeability were measured (see Table 4).

### Production Example 18

### Production of Deodorizing Nonwoven Fabric D18

A deodorizer-containing processing liquid with a solid content concentration of 10% was prepared using a CuO.SiO₂ complex oxide powder, a hydrotalcite powder, and a polyester binder, at a mass ratio of 10 parts of a CuO.SiO₂ complex oxide, 10 parts of hydrotalcite, and 10 parts of a polyester resin solid content. Next, the deodorizer-containing processing liquid was coated uniformly on nonwoven fabric sheet W1 by padding coating, such that the coating amount of a CuO.SiO₂ complex oxide became 10 g/m², and the coating amount of hydrotalcite became 10 g/m², followed by drying at 130° C. to produce a deodorizing nonwoven fabric D18, in which a deodorizer was bound uniformly to nonwoven fabric sheet W1 entirely from one side to the other side. With respect thereto, the basis weight and the thickness as well as the air permeability for a single layer and a 2-layer laminate were measured (see Table 4).

### Production Example 19

### Production of Deodorizing Nonwoven Fabric D19

A deodorizer-containing processing liquid with a solid content concentration of 10% was prepared using a zirconium phosphate powder, a CuO.SiO₂ complex oxide powder, Adipic acid dihydrazide (30%)-supported silica gel powder, and a polyester binder, at a mass ratio of 4 parts of zirconium phosphate, 4 parts of a CuO.SiO₂ complex oxide, 3 parts of Adipic acid dihydrazide (30%)-supported silica gel, and 6 parts of a polyester resin solid content. Next, the deodorizer-containing processing liquid was coated uniformly on nonwoven fabric sheet W2 by padding coating, such that the coating amount of zirconium phosphate became 4 g/m², the coating amount of a CuO.SiO₂ complex oxide became 4 g/m², and the coating amount of Adipic acid dihydrazide (30%)-supported silica gel became 3 g/m², followed by drying at 130° C. to produce a deodorizing nonwoven fabric D19, in which a deodorizer was bound uniformly to nonwoven fabric sheet W2entirely from one side to the other side. With respect thereto, the basis weight and the thickness as well as the air permeability for a single layer and a 2-layer laminate were measured (see Table 4).

### Production Example 20

### Production of Deodorizing Nonwoven Fabric D20

A deodorizer-containing processing liquid with a solid content concentration of 10% was prepared using an activated carbon powder, and a polyester binder, at a mass ratio of 8 parts of activated carbon, and 4 parts of a polyester resin solid content. Next, the deodorizer-containing processing liquid was coated uniformly on nonwoven fabric sheet W1 by padding coating, such that the coating amount of activated carbon became 8 g/m², followed by drying at 130° C. to produce a deodorizing nonwoven fabric D20, in which a deodorizer was bound uniformly to nonwoven fabric sheet W1 entirely from one side to the other side. With respect thereto, the basis weight and the thickness as well as the air permeability for a single layer and a 2-layer laminate were measured (see Table 4).

TABLE 3

| | | | | | | | | | Air |
|---|---|---|---|---|---|---|---|---|---|
| | | | | | | | | Air | permeability |
| | Deodorizing | | | Deodorizer | | Basis | | permeability | of 2-layer |
| | nonwoven | Base | | processed | Objective | weight | Thickness | (cm³/ | laminate |
| | fabric filter | material | Deodorizer | amount (g) | malodorous gas | (g/m²) | (mm) | (cm² · s)) | (cm³/(cm² · s)) |
| Production | D1 | W1 | Zirconium phosphate | 4 | Ammonia | 32 | 0.23 | 306 | 183 |
| Example 1 | | | CuO•SiO₂ complex oxide | 4 | Methyl mercaptan | | | | |
| Production | D2 | W1 | Zirconium phosphate | 3 | Ammonia | 29 | 0.21 | 401 | 220 |
| Example 2 | | | CuO•SiO₂ complex oxide | 3 | Methyl mercaptan | | | | |
| Production | D3 | W1 | Aluminum silicate | 4 | Ammonia | 31 | 0.23 | 335 | 190 |
| Example 3 | | | Hydrous zirconium oxide | 3 | Acetic acid | | | | |
| Production | D4 | W1 | Zirconium phosphate | 4 | Ammonia | 37 | 0.31 | 210 | 132 |
| Example 4 | | | CuO•SiO₂ complex oxide | 4 | Methyl mercaptan | | | | |
| | | | Adipic acid dihydrazide | 3 | Acetaldehyde | | | | |
| | | | (30%)-supported silica gel | | | | | | |
| Production | D5 | W1 | Aluminum silicate | 4 | Ammonia | 31 | 0.26 | 365 | 201 |
| Example 5 | | | Active zinc oxide | 3 | Acetic acid | | | | |
| Production | D6 | W1 | Hydrous zirconium oxide | 3 | Acetic acid | 29 | 0.23 | 412 | 225 |
| Example 6 | | | Adipic acid dihydrazide | 3 | Acetaldehyde | | | | |
| | | | (30%)-supported silica gel | | | | | | |
| Production | D7 | W1 | Amorphous zeolite | 4 | Ammonia | 31 | 0.23 | 358 | 187 |
| Example 7 | | | Hydrotalcite | 3 | Acetic acid | | | | |
| Production | D8 | W1 | Zirconium phosphate | 4 | Ammonia | 37 | 0.35 | 201 | 123 |
| Example 8 | | | CuO•SiO₂ complex oxide | 4 | Methyl mercaptan | | | | |
| | | | Hydrous zirconium oxide | 3 | Acetic acid | | | | |
| Production | D9 | W1 | Aluminum silicate | 4 | Ammonia | 35 | 0.32 | 188 | 98 |
| Example 9 | | | Active zinc oxide | 3 | Acetic acid | | | | |
| | | | Adipic acid dihydrazide | 3 | Acetaldehyde | | | | |
| | | | (30%)-supported silica gel | | | | | | |
| Production | D10 | W1 | CuO•SiO₂ complex oxide | 4 | Methyl mercaptan | 31 | 0.26 | 350 | 210 |
| Example | | | Hydrotalcite | 3 | Acetic acid | | | | |
| 10 | | | | | | | | | |

TABLE 4

| | | | | | | | | | Air |
|---|---|---|---|---|---|---|---|---|---|
| | | | | | | | | | permeability |
| | | | | | | | | Air | of 2-layer |
| | Deodorizing | | | Deodorizer | Objective | Basis | | permeability | laminate |
| | nonwoven | Base | | processed | malodorous | weight | Thickness | (cm³/ | (cm³/ |
| | fabric filter | material | Deodorizer | amount (g) | gas | (g/m²) | (mm) | (cm² · s)) | (cm² · s)) |
| Production | D11 | W2 | Zirconium phosphate | 4 | Ammonia | 29 | 0.22 | 450 | 289 |
| Example 11 | | | CuO•SiO₂ complex oxide | 4 | Methyl mercaptan | | | | |
| Production | D12 | W3 | Zirconium phosphate | 8 | Ammonia | 64 | 0.5 | 150 | |
| Example 12 | | | CuO•SiO₂ complex oxide | 8 | Methyl mercaptan | | | | |
| Production | D13 | W3 | Zirconium phosphate | 6 | Ammonia | 58 | 0.45 | 168 | |
| Example 13 | | | CuO•SiO₂ complex oxide | 6 | Methyl mercaptan | | | | |
| Production | D14 | W3 | Aluminum silicate | 8 | Ammonia | 61 | 0.44 | 148 | |
| Example 14 | | | Hydrous zirconium oxide | 6 | Acetic acid | | | | |
| Production | D15 | W3 | Aluminum silicate | 8 | Ammonia | 61 | 0.42 | 153 | |
| Example 15 | | | Active zinc oxide | 6 | Acetic acid | | | | |
| Production | D16 | W3 | Hydrous zirconium oxide | 6 | Acetic acid | 58 | 0.42 | 144 | |
| Example 16 | | | Adipic acid dihydrazide | 6 | Acetaldehyde | | | | |
| | | | (30%)-supported silica gel | | | | | | |
| Production | D17 | W3 | Amorphous zeolite | 8 | Ammonia | 61 | 0.43 | 140 | |
| Example 17 | | | Hydrotalcite | 6 | Acetic acid | | | | |
| Production | D18 | W1 | CuO•SiO₂ complex oxide | 10 | Methyl mercaptan | 50 | 0.35 | 88 | 40 |
| Example 18 | | | Hydrotalcite | 10 | Acetic acid | | | | |
| Production | D19 | W2 | Zirconium phosphate | 4 | Ammonia | 34 | 0.2 | 405 | 253 |
| Example 19 | | | CuO•SiO₂ complex oxide | 4 | Methyl mercaptan | | | | |
| | | | Adipic acid dihydrazide | 3 | Acetaldehyde | | | | |
| | | | (30%)-supported silica gel | | | | | | |
| Production | D20 | W1 | Activated carbon | 8 | Ammonia | 32 | 0.27 | 287 | 176 |
| Example 20 | | | | | | | | | |

4. Production of Deodorizing Mask

A deodorizing nonwoven fabric produced as above, a nonwoven fabric with a basis weight of 25 g/m² obtained using a polypropylene resin fiber by a spunbond method (hereinafter referred to as “nonwoven fabric W4”), and a nonwoven fabric with a basis weight of 25 g/m² obtained using a polypropylene resin fiber by a melt blown method (hereinafter referred to as “dust-tight nonwoven fabric L1”) were all cut to a size of 175 mm×165 mm. Using these, deodorizing masks having a spatial structure of an omega pleat were produced by a heretofore known production method and production apparatus, and subjected to various evaluations. The results are shown in Table 5 and Table 6.

### Example 1

### Production and Evaluation of Deodorizing Mask M1

From the outermost layer, nonwoven fabric W4, 2 sheets of deodorizing nonwoven fabric D1 obtained in Production Example 1, dust-tight nonwoven fabric L1, and nonwoven fabric W4 were layered one on another in the mentioned order, and then folded to form an omega pleat and in consequence a rectangle with a size of 175 mm×95 mm. Thereafter, the rims of a laminate were melt bonded with a heat sealer (150° C.) in a state where a nose wire was inserted in a predetermined position of the main part of a mask constituted with the laminate. Next, ear loops were formed by ultrasonic fusion at both the ends of the main part of a mask to obtain deodorizing mask M1 constituted with 5 layers of nonwoven fabric layers having a spatial structure with an omega pleat.

Thereafter, using the obtained deodorizing mask M1, an air permeability measurement on a mask main body, reduction rate measurements for ammonia and methyl mercaptan, which were malodorous components, a sensory test wearing a deodorizing mask, and a hedonic scale evaluation of an odor of a deodorizing mask itself were conducted. The results are shown in Table 5.

### Example 2

### Production and Evaluation of Deodorizing Mask M2

Deodorizing mask M2 constituted with 5 layers of nonwoven fabric layers was obtained by the same method as Example 1, except that deodorizing nonwoven fabric D2 obtained in Production Example 2 was used instead of deodorizing nonwoven fabric D1. Thereafter, the same evaluations as in Example 1 were conducted. The results are shown in Table 5.

### Example 3

### Production and Evaluation of Deodorizing Mask M3

Deodorizing mask M3 constituted with 5 layers of nonwoven fabric layers was obtained by the same method as Example 1, except that deodorizing nonwoven fabric D3 obtained in Production Example 3 was used instead of deodorizing nonwoven fabric D1. Thereafter, using the obtained deodorizing mask M3, an air permeability measurement on a mask main part, reduction rate measurements for ammonia and acetic acid, which were malodorous components, a sensory test wearing a deodorizing mask, and a hedonic scale evaluation of an odor of a deodorizing mask itself were conducted. The results are shown in Table 5.

### Example 4

### Production and Evaluation of Deodorizing Mask M4

Deodorizing mask M4 constituted with 5 layers of nonwoven fabric layers was obtained by the same method as Example 1, except that deodorizing nonwoven fabric D4 obtained in Production Example 4 was used instead of deodorizing nonwoven fabric D1. Thereafter, using the obtained deodorizing mask M4, an air permeability measurement on a mask main part, reduction rate measurements for ammonia, methyl mercaptan, and acetaldehyde, which were malodorous components, a sensory test wearing a deodorizing mask, and a hedonic scale evaluation of an odor of a deodorizing mask itself were conducted. The results are shown in Table 5.

### Example 5

### Production and Evaluation of Deodorizing Mask M5

Deodorizing mask M5 constituted with 5 layers of nonwoven fabric layers was obtained by the same method as Example 1, except that deodorizing nonwoven fabric D5 obtained in Production Example 5 was used instead of deodorizing nonwoven fabric D1. Thereafter, using the obtained deodorizing mask M5, an air permeability measurement on a mask main part, reduction rate measurements for ammonia and acetic acid, which were malodorous components, a sensory test wearing a deodorizing mask, and a hedonic scale evaluation of an odor of a deodorizing mask itself were conducted. The results are shown in Table 5.

### Example 6

### Production and Evaluation of Deodorizing Mask M6

Deodorizing mask M6 constituted with 5 layers of nonwoven fabric layers was obtained by the same method as Example 1, except that deodorizing nonwoven fabric D6 obtained in Production Example 6 was used instead of deodorizing nonwoven fabric D1. Thereafter, using the obtained deodorizing mask M6, an air permeability measurement on a mask main part, reduction rate measurements for acetic acid and acetaldehyde, which were malodorous components, a sensory test wearing a deodorizing mask, and a hedonic scale evaluation of an odor of a deodorizing mask itself were conducted. The results are shown in Table 5.

### Example 7

### Production and Evaluation of Deodorizing Mask M7

Deodorizing mask M7 constituted with 5 layers of nonwoven fabric layers was obtained by the same method as Example 1, except that deodorizing nonwoven fabric D7 obtained in Production Example 7 was used instead of deodorizing nonwoven fabric D1. Thereafter, using the obtained deodorizing mask M7, an air permeability measurement on a mask main part, reduction rate measurements for ammonia and acetic acid, which were malodorous components, a sensory test wearing a deodorizing mask, and a hedonic scale evaluation of an odor of a deodorizing mask itself were conducted. The results are shown in Table 5.

### Example 8

### Production and Evaluation of Deodorizing Mask M8

Deodorizing mask constituted with 5 layers of nonwoven fabric layers was obtained by the same method as Example 1, except that deodorizing nonwoven fabric D8 obtained in Production Example 8 was used instead of deodorizing nonwoven fabric D1.

Thereafter, using the obtained deodorizing mask M8, an air permeability measurement on a mask main part, reduction rate measurements for the respective malodorous components of ammonia, methyl mercaptan, and acetic acid, a sensory test wearing a deodorizing mask, and a hedonic scale evaluation of an odor of a deodorizing mask itself were conducted. The results are shown in Table 5.

### Example 9

### Production and Evaluation of Deodorizing Mask M9

Deodorizing mask M9 constituted with 5 layers of nonwoven fabric layers was obtained by the same method as Example 1, except that deodorizing nonwoven fabric D9 obtained in Production Example 9 was used instead of deodorizing nonwoven fabric D1. Thereafter, using the obtained deodorizing mask M9, an air permeability measurement on a mask main part, reduction rate measurements for ammonia, acetic acid, and acetaldehyde, which were malodorous components, a sensory test wearing a deodorizing mask, and a hedonic scale evaluation of an odor of a deodorizing mask itself were conducted. The results are shown in Table 5.

### Example 10

### Production and Evaluation of Deodorizing Mask M10

Deodorizing mask M10 constituted with 5 layers of nonwoven fabric layers was obtained by the same method as Example 1, except that deodorizing nonwoven fabric D10 obtained in Production Example 10 was used instead of deodorizing nonwoven fabric D1. Thereafter, using the obtained deodorizing mask M10, an air permeability measurement on a mask main part, reduction rate measurements for methyl mercaptan, and acetic acid, which were malodorous components, a sensory test wearing a deodorizing mask, and a hedonic scale evaluation of an odor of a deodorizing mask itself were conducted. The results are shown in Table 5.

### Comparative Example 1

### Production and Evaluation of Deodorizing Mask M11

From the outermost layer, nonwoven fabric W4, 2 sheets of deodorizing nonwoven fabric D11 obtained in Production Example 11, dust-tight nonwoven fabric L1, and nonwoven fabric W4 were layered one on another in the mentioned order, and then folded to form an omega pleat and in consequence a rectangle with a size of 175 mm×95 mm. Thereafter, the rims of a laminate were melt bonded with a heat sealer (250° C.) in a state where a nose wire was inserted in a predetermined position of the main part of a mask constituted with the laminate. Next, ear loops were formed by ultrasonic fusion at both the ends of the main part of a mask to obtain deodorizing mask M11 constituted with 5 layers of nonwoven fabric layers having a spatial structure with an omega pleat.

Thereafter, using the obtained deodorizing mask M11, an air permeability measurement on a mask main body, reduction rate measurements for ammonia and methyl mercaptan, which were malodorous components, a sensory test wearing a deodorizing mask, and a hedonic scale evaluation of an odor of a deodorizing mask itself were conducted. The results are shown in Table 6.

### Comparative Example 2

### Production and Evaluation of Deodorizing Mask M12

From the outermost layer, nonwoven fabric W4, deodorizing nonwoven fabric D12 obtained in Production Example 12, dust-tight nonwoven fabric L1, and nonwoven fabric W4 were layered one on another in the mentioned order, and then folded to form an omega pleat and in consequence a rectangle with a size of 175 mm×95 mm. Thereafter, the rims of a laminate were melt bonded with a heat sealer (150° C.) in a state where a nose wire was inserted in a predetermined position of the main part of a mask constituted with the laminate. Next, ear loops were formed by ultrasonic fusion at both the ends of the main part of a mask to obtain deodorizing mask M12 constituted with 4 layers of nonwoven fabric layers having a spatial structure with an omega pleat.

Thereafter, using the obtained deodorizing mask M12, an air permeability measurement on a mask main body, reduction rate measurements for ammonia and methyl mercaptan, which were malodorous components, a sensory test wearing a deodorizing mask, and a hedonic scale evaluation of an odor of a deodorizing mask itself were conducted. The results are shown in Table 6.

### Comparative Example 3

### Production and Evaluation of Deodorizing Mask M13

Deodorizing mask M13 constituted with 4 layers of nonwoven fabric layers was obtained by the same method as Comparative Example 2, except that deodorizing nonwoven fabric D13 obtained in Production Example 13 was used instead of deodorizing nonwoven fabric D12. Thereafter, using the obtained deodorizing mask M13, an air permeability measurement on a mask main part, reduction rate measurements for ammonia, and methyl mercaptan, which were malodorous components, a sensory test wearing a deodorizing mask, and a hedonic scale evaluation of an odor of a deodorizing mask itself were conducted. The results are shown in Table 6.

### Comparative Example 4

### Production and Evaluation of Deodorizing Mask M14

Deodorizing mask M14 constituted with 4 layers of nonwoven fabric layers was obtained by the same method as Comparative Example 2, except that deodorizing nonwoven fabric D14 obtained in Production Example 14 was used instead of deodorizing nonwoven fabric D12. Thereafter, using the obtained deodorizing mask M14, an air permeability measurement on a mask main part, reduction rate measurements for ammonia, and acetic acid, which were malodorous components, a sensory test wearing a deodorizing mask, and a hedonic scale evaluation of an odor of a deodorizing mask itself were conducted. The results are shown in Table 6.

### Comparative Example 5

### Production and Evaluation of Deodorizing Mask M15

Deodorizing mask M15 constituted with 4 layers of nonwoven fabric layers was obtained by the same method as Comparative Example 2, except that deodorizing nonwoven fabric D15 obtained in Production Example 15 was used instead of deodorizing nonwoven fabric D12. Thereafter, using the obtained deodorizing mask M15, an air permeability measurement on a mask main part, reduction rate measurements for ammonia, and acetic acid, which were malodorous components, a sensory test wearing a deodorizing mask, and a hedonic scale evaluation of an odor of a deodorizing mask itself were conducted. The results are shown in Table 6.

### Comparative Example 6

### Production and Evaluation of Deodorizing Mask M16

Deodorizing mask M16 constituted with 4 layers of nonwoven fabric layers was obtained by the same method as Comparative Example 2, except that deodorizing nonwoven fabric D16 obtained in Production Example 16 was used instead of deodorizing nonwoven fabric D12. Thereafter, using the obtained deodorizing mask M16, an air permeability measurement on a mask main part, reduction rate measurements for acetic acid, and acetaldehyde, which were malodorous components, a sensory test wearing a deodorizing mask, and a hedonic scale evaluation of an odor of a deodorizing mask itself were conducted. The results are shown in Table 6.

### Comparative Example 7

### Production and Evaluation of Deodorizing Mask M17

Deodorizing mask M17 constituted with 4 layers of nonwoven fabric layers was obtained by the same method as Comparative Example 2, except that deodorizing nonwoven fabric D17 obtained in Production Example 17 was used instead of deodorizing nonwoven fabric D12. Thereafter, using the obtained deodorizing mask M17, an air permeability measurement on a mask main part, reduction rate measurements for ammonia, and acetic acid, which were malodorous components, a sensory test wearing a deodorizing mask, and a hedonic scale evaluation of an odor of a deodorizing mask itself were conducted. The results are shown in Table 6.

### Comparative Example 8

### Production and Evaluation of Deodorizing Mask M18

Deodorizing mask M18 constituted with 5 layers of nonwoven fabric layers was obtained by the same method as Example 1, except that deodorizing nonwoven fabric D18 obtained in Production Example 18 was used instead of deodorizing nonwoven fabric D1. Thereafter, using the obtained deodorizing mask M18, an air permeability measurement on a mask main part, reduction rate measurements for methyl mercaptan, and acetic acid, which were malodorous components, a sensory test wearing a deodorizing mask, and a hedonic scale evaluation of an odor of a deodorizing mask itself were conducted. The results are shown in Table 6.

### Comparative Example 9

### Production and Evaluation of Deodorizing Mask M19

Deodorizing mask M19 constituted with 5 layers of nonwoven fabric layers was obtained by the same method as Example 1, except that deodorizing nonwoven fabric D19 obtained in Production Example 19 was used instead of deodorizing nonwoven fabric D1. Thereafter, using the obtained deodorizing mask M19, an air permeability measurement on a mask main part, reduction rate measurements for ammonia, methyl mercaptan, and acetaldehyde, which were malodorous components, a sensory test wearing a deodorizing mask, and a hedonic scale evaluation of an odor of a deodorizing mask itself were conducted. The results are shown in Table 6.

### Comparative Example 10

### Production and Evaluation of Deodorizing Mask M20

Deodorizing mask M20 constituted with 4 layers of nonwoven fabric layers was obtained by the same method as Comparative Example 2, except that deodorizing nonwoven fabric D1 obtained in Production Example 1 was used instead of deodorizing nonwoven fabric D12. Thereafter, using the obtained deodorizing mask M20, an air permeability measurement on a mask main part, reduction rate measurements for ammonia, and methyl mercaptan, which were malodorous components, a sensory test wearing a deodorizing mask, and a hedonic scale evaluation of an odor of a deodorizing mask itself were conducted. The results are shown in Table 6.

### Comparative Example 11

### Production and Evaluation of Deodorizing Mask M21

Deodorizing mask M20 constituted with 5 layers of nonwoven fabric layers was obtained by the same method as Example 1, except that deodorizing nonwoven fabric D20 obtained in Production Example 20 was used instead of deodorizing nonwoven fabric D1. Thereafter, using the obtained deodorizing mask M20, an air permeability measurement on a mask main part, reduction rate measurements for ammonia, which was a malodorous component, a sensory test wearing a deodorizing mask, and a hedonic scale evaluation of an odor of a deodorizing mask itself were conducted. The results are shown in Table 6.

TABLE 5

| | | | | Deodorizing performance | | |
|---|---|---|---|---|---|---|
| | | Air | | Malodorous | | |
| | Deodorizing | permeability | Objective | component | Odor intensity | Hedonic scale of |
| Example | mask | (cm³/(cm² · s)) | malodorous gas | reduction rate (%) | by sensory test | deodorizing mask |
| Example 1 | M1 | 31.5 | Ammonia | 97 | 0.5 | −0.5 |
| | | | Methyl mercaptan | 96 | 0.8 | |
| Example 2 | M2 | 32.4 | Ammonia | 92 | 0.8 | −0.3 |
| | | | Methyl mercaptan | 91 | 1.2 | |
| Example 3 | M3 | 32.3 | Ammonia | 91 | 0.8 | −0.2 |
| | | | Acetic acid | 95 | 0.5 | |
| Example 4 | M4 | 30.5 | Ammonia | 96 | 0.5 | −0.7 |
| | | | Methyl mercaptan | 92 | 1.0 | |
| | | | Acetaldehyde | 90 | 1.0 | |
| Example 5 | M5 | 32.7 | Ammonia | 92 | 1.0 | −0.5 |
| | | | Acetic acid | 90 | 0.8 | |
| Example 6 | M6 | 33.0 | Acetic acid | 92 | 0.3 | −0.5 |
| | | | Acetaldehyde | 91 | 0.7 | |
| Example 7 | M7 | 31.5 | Ammonia | 96 | 0.5 | −0.3 |
| | | | Acetic acid | 96 | 0.3 | |
| Example 8 | M8 | 30.1 | Ammonia | 96 | 0.6 | −0.7 |
| | | | Methyl mercaptan | 95 | 0.8 | |
| | | | Acetic acid | 92 | 0.8 | |
| Example 9 | M9 | 31.0 | Ammonia | 91 | 0.8 | −0.5 |
| | | | Acetic acid | 91 | 0.7 | |
| | | | Acetaldehyde | 90 | 0.7 | |
| Example 10 | M10 | 33.1 | Methyl mercaptan | 94 | 0.5 | −0.5 |
| | | | Acetic acid | 96 | 0.3 | |

TABLE 6

| | | | | Deodorizing performance | | |
|---|---|---|---|---|---|---|
| | | Air | | Malodorous | | |
| Comparative | Deodorizing | permeability | Objective | component | Odor intensity | Hedonic scale of |
| Example | mask | (cm³/(cm² · s)) | malodorous gas | reduction rate (%) | by sensory test | deodorizing mask |
| Comparative | M11 | 34.3 | Ammonia | 94 | 0.6 | −2.0 |
| Example 1 | | | Methyl mercaptan | 93 | 0.8 | |
| Comparative | M12 | 25.4 | Ammonia | 79 | 1.4 | −1.0 |
| Example 2 | | | Methyl mercaptan | 75 | 1.5 | |
| Comparative | M13 | 26.7 | Ammonia | 75 | 1.3 | −1.0 |
| Example | | | Methyl mercaptan | 71 | 1.9 | |
| Comparative | M14 | 25.3 | Ammonia | 69 | 1.2 | −1.0 |
| Example 4 | | | Acetic acid | 78 | 0.9 | |
| Comparative | M15 | 26.8 | Ammonia | 68 | 0.9 | −1.0 |
| Example 5 | | | Acetic acid | 71 | 1.2 | |
| Comparative | M16 | 25.5 | Acetic acid | 75 | 0.7 | −0.8 |
| Example 6 | | | Acetaldehyde | 70 | 1.5 | |
| Comparative | M17 | 25.7 | Ammonia | 78 | 0.7 | −0.8 |
| Example 7 | | | Acetic acid | 79 | 0.5 | |
| Comparative | M18 | 16.3 | Methyl mercaptan | 95 | 0.5 | −1.2 |
| Example 8 | | | Acetic acid | 96 | 0.3 | |
| Comparative | M19 | 31.1 | Ammonia | 85 | 0.8 | −2.5 |
| Example 9 | | | Methyl mercaptan | 81 | 1.8 | |
| | | | Acetaldehyde | 85 | 1.0 | |
| Comparative | M20 | 35.6 | Ammonia | 60 | 2.5 | −0.3 |
| Example 10 | | | Methyl mercaptan | 55 | 3.0 | |
| Comparative | M21 | 30.9 | Ammonia | 38 | 4.5 | −0.5 |
| Example 11 | | | | | | |

In all of Examples 1 to 10, high deodorizing performance with a malodorous component reduction rate of 90% or higher was exhibited, and a malodor could be reduced to almost odorless level equivalent to odor intensity of 1.2 or less by a sensory test. Except that the deodorizing nonwoven fabric layers of Comparative Examples 2, 3, 4, 5, 6, and 7 were of 1 layer, and that the deodorizing nonwoven fabric layers of Examples 1, 2, 3, 5, 6, and 7 were of 2 layers, the material and production method of a nonwoven fabric, the composition and processed amount of a chemisorption deodorizer and a binder, and the basis weight with respect to the respective deodorizing nonwoven fabric layers were identical, however in Comparative Examples the malodorous component reduction rate, and sensory test odor intensity, as well as deodorizing performance were inferior, and further the air permeability of a mask was also inferior, which demonstrates effectiveness of duplication of a deodorizing nonwoven fabric layer.

In all of Examples 1 through 10, the hedonic scale of an odor of a mask itself was −1 or better. In contrast, in Comparative Example 1 and Comparative Example 9, namely examples where a base cloth of a nonwoven fabric did not contain polyethylene as a component, their hedonic scale was -2 or worse indicating grown unpleasantness.

Comparative Example 8 is an example where a deodorizing nonwoven fabric layer with an air permeability of 50 cm³/(cm²·s) or less was used for a mask. In this case, although deodorizing performance was high, the air permeability of a mask was so low that the mask was of no practical use. In Comparative Example 10, a deodorizing filter used in Example 1 was used in a single layer. In this case, the deodorizing performance in terms of both the malodorous component reduction rate and the odor intensity by a sensory test was inferior. Further, in Comparative Example 11, namely an example where activated carbon was used as a deodorizer other than a chemisorption-type deodorizer, the deodorizing performance in terms of both the malodorous component reduction rate and the odor intensity by a sensory test was significantly poor.

### INDUSTRIAL APPLICABILITY

With a deodorizing mask of the invention, for example, with respect to a malodorous gas, such as an excretion odor and a putrid odor, high deodorizing performance may be obtained in a moment on a malodorous component passing through a deodorizing fiber layer. Therefore, the same may be utilized effectively at locations where a malodor is emitted during operation at various working sites, such as an excrement treatment plant, a livestock farm, a sewage treatment plant, a sanitation facility, a garbage treatment plant, a fertilizer plant, a chemical plant, a hospital, a nursing facility, a fishing port, and a disaster-stricken spot, or at home.

### DESCRIPTION OF REFERENCE NUMERALS AND SIGNS

1: Mask main body
2: Upper part of mask main body
3: Ear loop
4: Nose wire
5: Heat sealed seam part
6: Heat sealed mesh part
7: Open air side PP nonwoven fabric layer
8: Deodorizing nonwoven fabric layer (deodorizing fiber layer)
9: Dust-tight nonwoven fabric layer
10: Facial side PP nonwoven fabric layer
11: Pleat

1. A deodorizing filter for a mask comprising 2 or more layers of deodorizing fiber layers comprising a fiber and a chemisorption-type deodorizer, wherein:
the deodorizing fiber layer contains a polyethylene resin fiber, and
the thickness of the deodorizing fiber layer is from 0.15 to 0.4 mm, and the basis weight of the deodorizing fiber layer is from 20 to 45 g/m².

2. The deodorizing filter for a mask according to claim 1, wherein a base material for the deodorizing filter for a mask is a nonwoven fabric.

3. The deodorizing filter for a mask according to claim 1, wherein any of the 2 or more layers of deodorizing fiber layers contain the same chemisorption-type deodorizer.

4. The deodorizing filter for a mask according to claim 1, wherein the chemisorption-type deodorizer is a compound selected out of the group consisting of [1] a phosphate of a tetravalent metal, [2] an amine compound, [3] zeolite, [4] an amorphous complex oxide expressed by X₂O—Al₂O₃—SiO₂ (X is at least one kind of atom selected out of the group consisting of Na, K, and Li), [5] a compound containing at least one kind of atom selected out of the group consisting of Ag, Cu, Zn, and Mn, [6] at least one kind of zirconium compound selected out of the group consisting of hydrated zirconium oxide and zirconium oxide, [7] a hydrotalcite compound, and [8] an amorphous active oxide.

5. The deodorizing filter for a mask according to claim 1, wherein the content of a chemisorption-type deodorizer in the deodorizing fiber layer is 1 g/m² or more.

6. The deodorizing filter for a mask according to claim 1, wherein the deodorizing fiber layer is produced by immersing a fiber in a liquid containing a chemisorption-type deodorizer, and then drying the same.

7. The deodorizing filter for a mask according to claim 1, wherein a chemisorption-type deodorizer is bonded to a fiber by a binder in the deodorizing fiber layer.

8. The deodorizing filter for a mask according to claim 1, wherein the air permeability according to a Frazier type method is from 50 to 350 cm³/(cm²·s).

9. A deodorizing mask comprising as a laminate the deodorizing filter for a mask according to claim 1 and a filter other than the deodorizing filter.
